(19) United States
(12) Patent Application Publication
(10) Pub. No.: US 2026/0100284 A1
(43) Pub. Date: Apr. 9, 2026

(54) DOMAIN EXTENSION LEARNING DEVICE, DOMAIN EXTENSION LEARNING METHOD, AND RECORDING MEDIUM

(71) Applicant: NEC Corporation, Tokyo (JP)

(72) Inventors: Kensuke WAGATA, Tokyo (JP); Chenhui HUANG, Tokyo (JP); Fumiyuki NIHEY, Tokyo (JP)

(73) Assignee: NEC Corporation, Tokyo (JP)

(21) Appl. No.: 19/339,426

(22) Filed: Sep. 25, 2025

(30) Foreign Application Priority Data
Oct. 7, 2024 (JP) 2024-175519

(51) Int. Cl. G16H 50/50; G06N 20/00

(52) U.S. Cl. CPC G16H 50/50; G06N 20/00

(57) ABSTRACT

In a domain extension learning device, a generation means generates pseudo medical examination data. A prediction means predicts a domain from the pseudo medical examination data. A calculation means calculates the difference between the predicted domain and the specified domain. An update means updates the parameters of the generation means based on the difference. The generation means may comprise a deep learning model. According to the domain extension learning device, it is possible to generate pseudo data of an unknown domain. As a result, the user can acquire learning data including a wide variety of domains, and can optimize a disease risk prediction model. Furthermore, by using this disease risk prediction model, it is possible to support the user's decision making.

### INCORPORATION BY REFERENCE

This application is based upon and claims the benefit of priority from Japanese patent application No. 2024-175519, filed on October 7, 2024, the disclosure of which is incorporated herein in its entirety by reference.

### TECHNICAL FIELD

The present disclosure relates to a data generation technique.

### BACKGROUND ART

In recent years, utilization of big data has progressed. For example, big data is utilized to perform highly accurate prediction by artificial intelligence (AI). However, collection of big data requires a monetary cost and a time cost. On the other hand, Patent Document 1 discloses a method of generating pseudo data for use in learning of a model.

Patent Document 1: Japanese Patent 7402359

### SUMMARY

However, even with the method of Patent Document 1, a wide variety of data cannot be generated.

One object of the present disclosure is to provide a domain extension learning device capable of generating pseudo data of an unknown domain.

According to an example aspect of the present invention, there is provided a domain extension learning device, including:

at least one memory configured to store instructions; and

at least one processor configured to execute the instructions to:

generate pseudo medical examination data using a model targeted for training;

predict a domain from the pseudo medical examination data;

calculate a difference between the predicted domain and a specified domain; and

update a parameter of the model based on the difference.

According to another example aspect of the present invention, there is provided a domain extension learning method including:

generating pseudo medical examination data using a model targeted for training;

predicting a domain from the pseudo medical examination data;

calculating a difference between the predicted domain and a specified domain; and

updating a parameter of the model based on the difference.

According to a further example aspect of the present invention, there is provided a recording medium recording a program for causing a computer to execute processing including:

generating pseudo medical examination data using a model targeted for training;

predicting a domain from the pseudo medical examination data;

calculating a difference between the predicted domain and a specified domain; and

updating a parameter of the model based on the difference.

### EFFECT

According to the present disclosure, it is possible to provide a domain extension learning device capable of generating pseudo data of an unknown domain.

### BRIEF DESCRIPTION OF THE DRAWINGS

FIG. 1 is a block diagram illustrating a hardware configuration of a learning device according to the present disclosure;

FIG. 2 is a block diagram illustrating a functional configuration of the learning device according to the present disclosure;

FIG. 3 illustrates a usage example of a learned pseudo data generation unit;

FIG. 4 is a flowchart of processing by the learning device according to the present disclosure;

FIG. 5 is a block diagram illustrating a functional configuration of another learning device according to the present disclosure;

FIG. 6 illustrates a usage example of another learned pseudo data generation unit;

FIG. 7 is a flowchart of processing by the another learning device according to the present disclosure;

FIG. 8 is a block diagram illustrating a functional configuration of the another learning device according the present disclosure; and

FIG. 9 is a flowchart of processing by the another learning device according to the present disclosure.

### EXAMPLE EMBODIMENT

Hereinafter, preferred example embodiments of the present disclosure will be described with reference to the drawings.

### First Example Embodiment

### Outline Description

In recent years, in the field of medical healthcare, a disease risk prediction model utilizing big data such as medical examination data has been developed. The prediction model predicts a disease risk for a patient having various attributes (hereinafter also referred to as a “domain”) such as race, gender, disease, age, and blood pressure value. In order to perform highly accurate prediction for patients having various domains, learning data including a wide variety of domains is required. However, it is unrealistic to exhaustively collect the learning data as described above from the viewpoint of cost, data privacy, a difference in data format for each hospital, and the like.

Therefore, in the present example embodiment, a trained model that generates the pseudo medical examination data of the domain specified by the user is generated. At this time, the user can specify a domain that is not included in the collected actual medical examination data. As a result, it is possible to generate data of a range that is not covered by the collected actual medical examination data.

In the present example embodiment, the collected actual medical examination data is also referred to as “actual data”, and the pseudo medical examination data generated by the learning model is also referred to as “pseudo data”.

In the present example embodiment, the domain included in the actual data is also referred to as a “known domain”, and the domain not included in the actual data is also referred to as an “unknown domain”. For example, in a case where there is no data of a 30 year-old patient in the actual data, “30 years old” is an unknown domain.

### Hardware Configuration

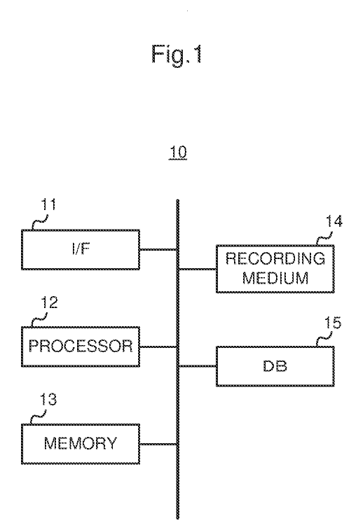

FIG. 1 is a block diagram illustrating a hardware configuration of a learning device 10 according to the first example embodiment. The learning device 10 is an example of a domain extension learning device. As illustrated, the learning device 10 includes an interface (I/F) 11, a processor 12, a memory 13, a recording medium 14, and a database (DB) 15.

The I/F 11 inputs and outputs data to and from an external device. Specifically, the I/F 11 acquires learning data used by the learning device 10 from an external device.

The processor 12 is a computer such as a central processing unit (CPU), and takes overall control of the learning device 10 by executing a program prepared in advance. As the processor 12, for example, a graphics processing unit (GPU), a digital signal processor (DSP), a micro processing unit (MPU), a floating point number processing unit (FPU), a physics processing unit (PPU), a tensor processing unit (TPU), a quantum processor, a combination thereof, or the like may be used. The processor 12 executes training processing to be described later.

The memory 13 includes a read only memory (ROM), a random access memory (RAM), and the like. The memory 13 stores a model of a deep neural network (DNN) used by the learning device 10, and the like. The memory 13 is also used as a work memory during execution of various types of processing by the processor 12.

The recording medium 14 is a non-volatile non-transitory recording medium such as a disk-shaped recording medium or a semiconductor memory, and is attachable to and detachable from the learning device 10. The recording medium 14 records various programs executed by the processor 12. In a case where the learning device 10 executes various types of processing, a program recorded in the recording medium 14 is loaded into the memory 13 and executed by the processor 12. The DB 15 stores data input via the I/F 11.

In addition to the above, the learning device 10 may include a display device such as a liquid crystal display and an input device such as a keyboard and a mouse. The display device and input device are used by an administrator of the learning device 10 to perform required administration, for example.

### Functional Configuration

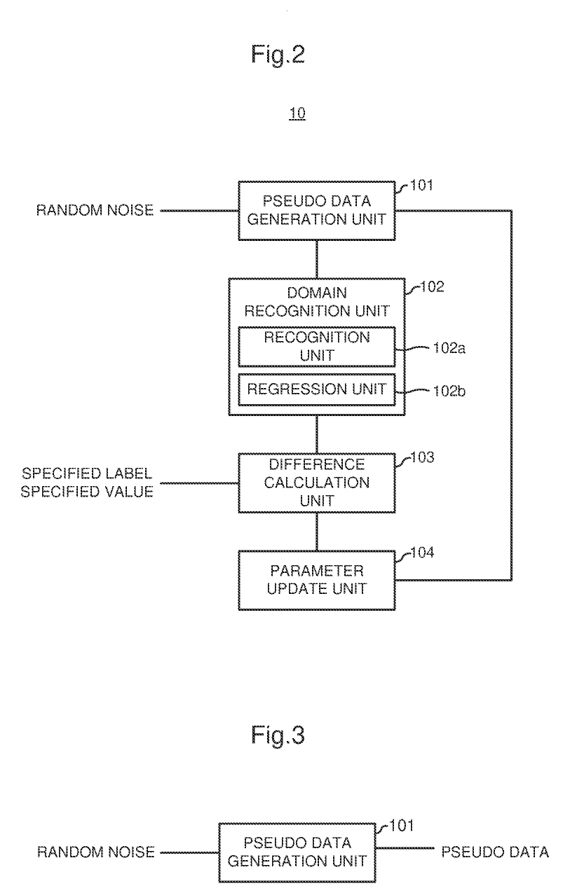

FIG. 2 is a block diagram illustrating a functional configuration of the learning device 10 according to the first example embodiment. The learning device 10 functionally includes a pseudo data generation unit 101, a domain recognition unit 102, a difference calculation unit 103, and a parameter update unit 104. Note that the pseudo data generation unit 101 is a target for training and includes a DNN and the like.

The random noise, the specified label, and the specified value are input to the learning device 10 via the I/F 11. The random noise is input to the pseudo data generation unit 101. The specified label and the specified value are input to the difference calculation unit 103.

The specified label and the specified value are domains provided by the user. The learning device 10 trains the pseudo data generation unit 101 to generate the pseudo data of the domain given as the specified label and the specified value. For example, in a case where it is desired to generate pseudo data of “with diabetes” and “25 years old”, the user sets “probability of presence or absence of diabetes {1, 0}” as the specified label and sets “age {25}” as the specified value. Note that the specified label is not limited to the one hot label, and may be a soft label such as “probability of presence or absence of diabetes {0.8, 0.2}”. In addition, a known domain may be set as the specified value, or an unknown domain may be set as the specified value.

The pseudo data generation unit 101 generates pseudo data from the random noise. The pseudo data is fictitious medical examination data and has the same item as the actual data. Examples of the item include systolic blood pressure, diastolic blood pressure, fasting blood glucose, and γ-GTP. The pseudo data generation unit 101 outputs the pseudo data to the domain recognition unit 102.

The domain recognition unit 102 includes a recognition unit 102a and a regression unit 102b. The recognition unit 102a includes a classifier trained in advance with actual data, an abnormality detection model, and the like. In addition, the regression unit 102b is configured by a regression device trained in advance with actual data.

The recognition unit 102a predicts race, gender, disease, and the like for the input pseudo data, and outputs a prediction label to the difference calculation unit 103. For example, the recognition unit 102a classifies the pseudo data and outputs the result as a probability value. The recognition unit 102a sets the output result as a prediction label. Hereinafter, an example of output by the recognition unit 102a will be described.

### Output example 1 race: probability value of Japanese, American 0.2, 0.8

### Output example 2 presence or absence of disease: probability value 0.8 of with diabetes

The regression unit 102b predicts age, blood pressure value, BMI, and the like for the input pseudo data, and outputs the prediction value to the difference calculation unit 103. For example, the regression unit 102b outputs a scalar value (for example, 25) representing age or a scalar value (for example, 120) representing a blood pressure value. The regression unit 102b uses these scalar values as prediction values.

Note that the domains predicted by the recognition unit 102a and the regression unit 102b are determined in advance based on the specified label and the specified value. For example, in a case where the probability of presence or absence of diabetes is set as the specified label, the recognition unit 102a predicts the presence or absence of diabetes with respect to the input pseudo data. In addition, in a case where age is set as the specified value, the regression unit 102b predicts age for the input pseudo data.

The difference calculation unit 103 calculates a difference between the specified label and the prediction label and a difference between the specified value and the prediction value. The difference calculation unit 103 calculates the difference between the specified label and the prediction label by using, for example, a method such as cross entropy, cross entropy with temperature, KL divergence, L1 distance, or L2 distance. In addition, the difference calculation unit 103 calculates, for example, a mean square error between the specified value and the prediction value or a mean absolute error between the specified value and the prediction value as the difference between the specified value and the prediction value.

The difference calculation unit 103 adds the difference between the specified label and the prediction label and the difference between the specified value and the prediction value, and outputs the sum to the parameter update unit 104.

The parameter update unit 104 optimizes the parameters of the DNN included in the pseudo data generation unit 101 in such a way that the sum value of the difference between the specified label and the prediction label and the difference between the specified value and the prediction value is minimized. In this manner, the training of the pseudo data generation unit 101 is performed in such a way as to generate the pseudo data of the specified domain.

For example, in a case where “probability of presence or absence of diabetes {1, 0}, age {25}” is given as the specified label and the specified value, the pseudo data generation unit 101 generates the pseudo data in such a way that the recognition unit 102a predicts that there is diabetes and the regression unit 102b predicts that the age is 25 in order to minimize the difference between the specified label and the specified value and the prediction label and the prediction value. As a result, the pseudo data generation unit 101 can arbitrarily generate pseudo data of 25 years old with diabetes.

### Generation of Pseudo Data

FIG. 3 illustrates a usage example of a trained pseudo data generation unit 101. As illustrated in FIG. 3, the trained pseudo data generation unit 101 generates pseudo data with random noise as an input.

By performing the training as described above, the pseudo data generation unit 101 can generate pseudo data of an unknown domain that is not covered by actual data, and the user can acquire data including a wide variety of domains.

In the above configuration, the pseudo data generation unit 101 is an example of a generation means, the domain recognition unit 102 is an example of a prediction means, the difference calculation unit 103 is an example of a calculation means, and the parameter update unit 104 is an example of an update means.

### Training Processing

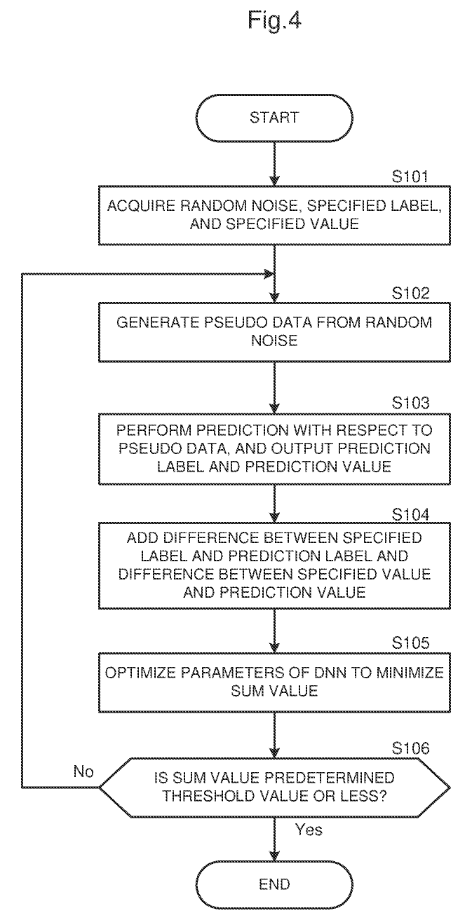

Next, training processing in which training as described above is performed will be described. FIG. 4 is a flowchart of training processing by the learning device 10. This processing is achieved by the processor 12 illustrated in FIG. 1 executing a program prepared in advance and operating as each element illustrated in FIG. 2.

First, the random noise, the specified label, and the specified value are input to the learning device 10 via the I/F 11 (step S101). The random noise is input to the pseudo data generation unit 101. The specified label and the specified value are input to the difference calculation unit 103.

Next, the pseudo data generation unit 101 generates pseudo data from the random noise (step S102). The pseudo data generation unit 101 outputs the pseudo data to the domain recognition unit 102. Next, the domain recognition unit 102 performs prediction with respect to the input pseudo data, and outputs the prediction label and the prediction value to the difference calculation unit 103 (step S103).

Next, the difference calculation unit 103 adds the difference between the specified label and the prediction label and the difference between the specified value and the prediction value, and outputs the sum to the parameter update unit 104 (step S104). Next, the parameter update unit 104 optimizes the parameters of the DNN included in the pseudo data generation unit 101 in such a way that the sum value of the difference between the specified label and the prediction label and the difference between the specified value and the prediction value is minimized (step S105). The processing of steps S102 to S105 is repeatedly executed, and for example, in a case where the sum value becomes equal to or less than a predetermined threshold value (step S106: Yes), the processing ends.

### Modification

Next, a modification of the first example embodiment will be described.

Although the medical examination data has been described above as an example, the data generated by the pseudo data generation unit 101 is not limited to this. The learning device of the present example embodiment can be applied to tabular data including items and their values in addition to the medical examination data. For example, the learning device of the present example embodiment may train the pseudo data generation unit 101 in such a way as to generate the diagnostic data of the machine. In this case, the domain includes voltage, damage, oil leakage, and the like.

### Second Example Embodiment

Next, a second example embodiment will be described. In the second example embodiment, the trained model that generates the pseudo data is generated using the actual data and the domain conversion label as inputs. The user can specify a domain to be generated by using the domain conversion label.

A learning device 20 according to the second example embodiment has a hardware configuration similar to that of the learning device 10 according to the first example embodiment, and thus description thereof will be omitted. The learning device 20 is an example of a domain extension learning device.

In addition, the domain of the second example embodiment includes a combination of a plurality of domains. For example, in the second example embodiment, each of “40 years old, Japanese, with diabetes” and “30 years old, without diabetes” is treated as one domain. In addition, the domain of the second example embodiment includes at least one continuous variable and an arbitrary number of category variables.

### Functional Configuration

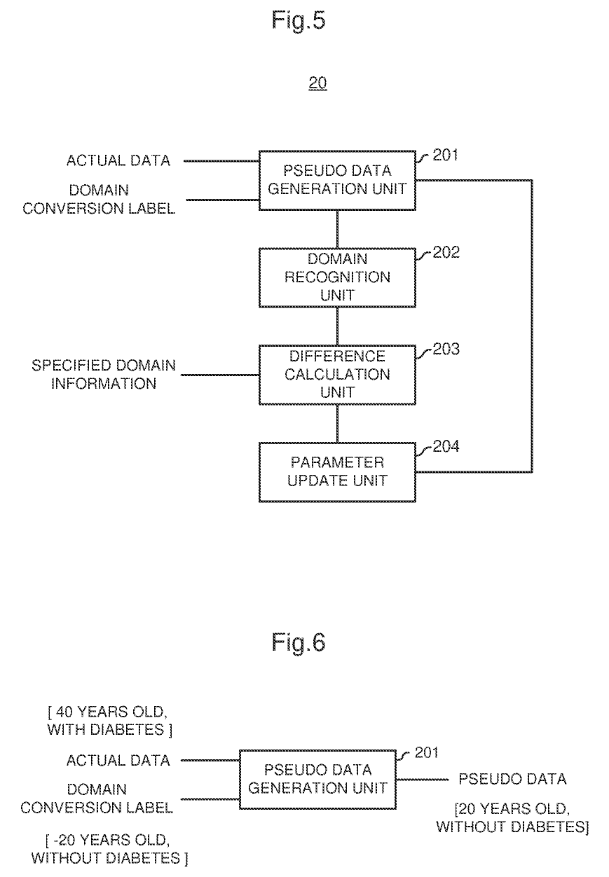

FIG. 5 is a block diagram illustrating a functional configuration of the learning device 20 according to the second example embodiment. The learning device 20 functionally includes a pseudo data generation unit 201, a domain recognition unit 202, a difference calculation unit 203, and a parameter update unit 204. The pseudo data generation unit 201 is a target for training and configured by a DNN and the like.

The actual data, the domain conversion label, and the specified domain information are input to the learning device 20 via the I/F 11. The actual data and the domain conversion label are input to the pseudo data generation unit 201. The specified domain information is input to the difference calculation unit 203.

The domain conversion label is a label indicating a difference between the domain of the target pseudo data (conversion destination) and the domain of the actual data (conversion source). At the time of training by the pseudo data generation unit 201, a known domain is set as a conversion destination.

Specifically, the domain conversion label is represented by a difference between a conversion destination and a conversion source continuous variable (such as age and BMI) and a conversion destination category variable (race, gender, disease, etc.). The domain conversion label is set to include a difference of at least one continuous variable. For example, in a case where the user desires to generate the pseudo data of “30 years old, without diabetes” from the actual data of “40 years old, with diabetes”, the user sets “-10, 0” as the domain conversion label. Here, “-10” represents -10 years old, which is a difference between 30 years old and 40 years old. In addition, “0” is a label indicating no diabetes. The learning device 20 trains the pseudo data generation unit 201 to convert the actual data into the pseudo data based on the actual data and the domain conversion label.

The specified domain information is a representative feature amount of the domain of the conversion destination. For example, in a case where the user desires to generate pseudo data of “30 years old, without diabetes”, the user sets an average value or a median value of feature amounts of actual data belonging to the domain as a representative feature amount. Furthermore, the specified domain information may be a label representing a domain of a conversion destination. For example, it is assumed that a label representing “30 years old, without diabetes” is “0” and a label representing “30 years old, with diabetes” is “1”. In a case where the user desires to generate pseudo data of “30 years old, with diabetes”, the user sets a label “1” representing “30 years old, with diabetes” as the specified domain information.

The pseudo data generation unit 201 generates pseudo data from the actual data and the domain conversion label. The pseudo data generation unit 201 outputs the generated pseudo data to the domain recognition unit 202.

The domain recognition unit 202 includes a feature amount extractor or a classifier that is pre-trained to recognize a domain from actual data.

In a case where the feature amount is given as the specified domain information, the domain recognition unit 202 extracts the feature amount from the input pseudo data and outputs the extracted feature amount to the difference calculation unit 203 as the prediction domain information. The domain recognition unit 202 outputs, for example, a 128 dimensional feature amount vector. On the other hand, in a case where a label is given as the specified domain information, the domain recognition unit 202 outputs the attribution probability value for each label from the input pseudo data, and outputs the attribution probability value for each label to the difference calculation unit 203 as the prediction domain information. For example, the domain recognition unit 202 outputs “0.2, 0.8” and the like as the attribution probability value of the label 0 (30 years old, without diabetes) and the label 1 (30 years old, with diabetes).

The difference calculation unit 203 calculates a difference between the specified domain information and the prediction domain information, and outputs the difference to the parameter update unit 204.

In a case where the feature amount is given as the specified domain information, the difference calculation unit 203 calculates a difference between the specified domain information and the prediction domain information by using a method such as cosine similarity, L1 distance, L2 distance, Chebyshev distance, or Minkowski distance. On the other hand, in a case where a domain label is given as the specified domain information, the difference calculation unit 203 calculates a difference between the specified domain information and the prediction domain information by using a method such as cross entropy, cross entropy with temperature, KL divergence, L1 distance, or L2 distance.

The parameter update unit 204 optimizes the parameters of the DNN included in the pseudo data generation unit 201 in such a way that the difference between the specified domain information and the prediction domain information is minimized. In this manner, the training of the pseudo data generation unit 201 is performed in such a way as to generate the pseudo data of the specified domain.

### Generation of Pseudo Data

FIG. 6 illustrates a usage example of a trained pseudo data generation unit 201. As illustrated in FIG. 6, the trained pseudo data generation unit 201 generates the pseudo data using the actual data and the domain conversion label as inputs.

Note that the user sets the known domain as the domain of the conversion destination at the time of training by the pseudo data generation unit 201, but can set the known domain or the unknown domain as the domain of the conversion destination at the time of data generation by the trained pseudo data generation unit 201. For example, in a case where the value range of the age of the actual data is 60's to 90's, the user can set less than 60, which is an unknown domain, as the domain of the conversion destination at the time of data generation.

As a result, the pseudo data generation unit 201 can generate pseudo data of an unknown domain that is not covered by actual data, and the user can acquire data including a wide variety of domains.

In the above configuration, the pseudo data generation unit 201 is an example of a generation means, the domain recognition unit 202 is an example of a prediction means, the difference calculation unit 203 is an example of a calculation means, and the parameter update unit 204 is an example of an update means.

### Training Processing

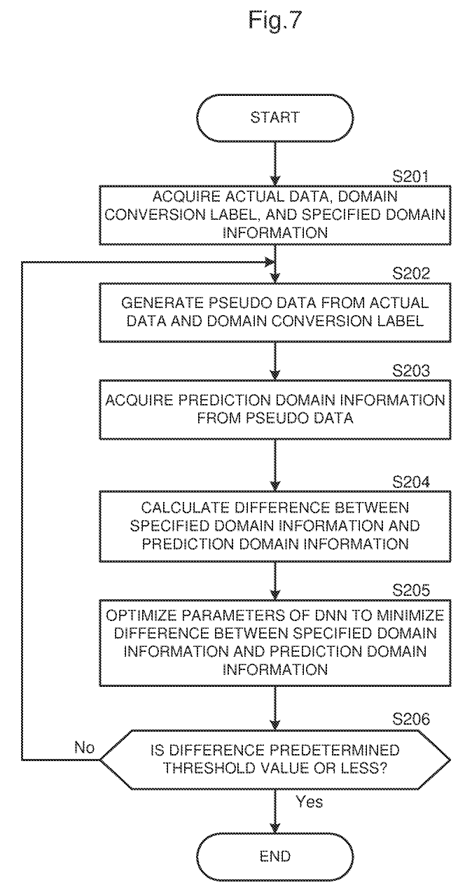

Next, training processing in which training as described above is performed will be described. FIG. 7 is a flowchart of training processing by the learning device 20. This processing is achieved by the processor 12 illustrated in FIG. 1 executing a program prepared in advance and operating as each element illustrated in FIG. 5.

First, the actual data, the domain conversion label, and the specified domain information are input to the learning device 20 via the I/F 11 (step S201). The actual data and the domain conversion label are input to the pseudo data generation unit 201. The specified domain information is input to the difference calculation unit 203.

Next, the pseudo data generation unit 201 generates pseudo data from the actual data and the domain conversion label (step S202). The pseudo data generation unit 201 outputs the generated pseudo data to the domain recognition unit 202. Next, the domain recognition unit 202 acquires prediction domain information from the input pseudo data and outputs the prediction domain information to the difference calculation unit 203 (step S203). Next, the difference calculation unit 203 calculates a difference between the specified domain information and the prediction domain information, and outputs the difference to the parameter update unit 204 (step S204).

Next, the parameter update unit 204 optimizes the parameters of the DNN constituting the pseudo data generation unit 201 in such a way that the difference between the specified domain information and the prediction domain information is minimized (step S205). The processing of steps S202 to S205 is repeatedly executed, and for example, in a case where the difference becomes equal to or less than a predetermined threshold value (step S206: Yes), the processing ends.

### Modification

Next, a modification of the second example embodiment will be described.

Although the medical examination data has been described above as an example, the data generated by the pseudo data generation unit 201 is not limited to this. The learning device of the present example embodiment can be applied to tabular data including items and their values in addition to the medical examination data. For example, the learning device of the present example embodiment may train the pseudo data generation unit 201 in such a way as to generate the diagnostic data of the machine. In this case, the domain includes voltage, damage, oil leakage, a combination of these, and the like.

### Third Example Embodiment

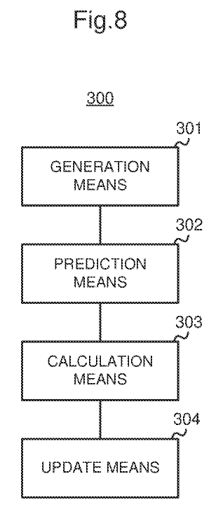

FIG. 8 is a block diagram illustrating a functional configuration of the domain extension learning device according to the third example embodiment. The domain extension learning device 300 includes a generation means 301, a prediction means 302, a calculation means 303, and an update means 304.

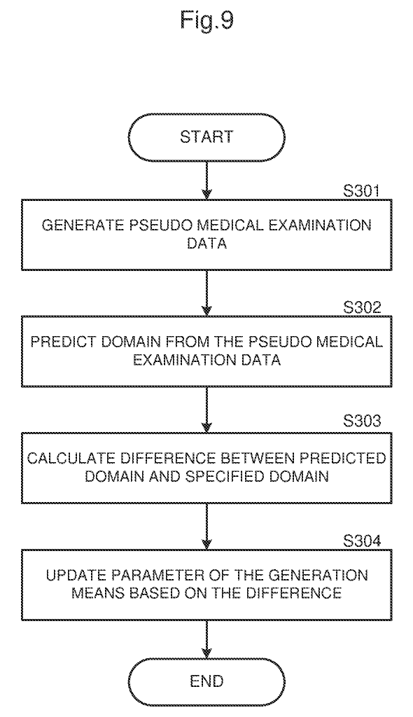

FIG. 9 is a flowchart of processing by a domain extension learning device according to the third example embodiment. The generation means 301 generates pseudo medical examination data (step S301). The prediction means 302 predicts a domain from the pseudo medical examination data (step S302). The calculation means 303 calculates the difference between the predicted domain and the specified domain (step S303). The update means 304 updates the parameters of the generation means based on the difference (step S304).

According to the domain extension learning device 300 of the third example embodiment, it is possible to generate pseudo data of an unknown domain. As a result, the user can acquire learning data including a wide variety of domains, and can optimize a disease risk prediction model.

Furthermore, by using this disease risk prediction model to predict disease risks and other related factors, it is possible to support the user's decision making regarding their health.

Some or all of the above example embodiments may also be described as the following Supplementary Notes, but are not limited to the following Supplementary Notes.

### Supplementary note 1

A domain extension learning device comprising:

a generation means for generating pseudo medical examination data;

a prediction means for predicting a domain from the pseudo medical examination data;

a calculation means for calculating a difference between the predicted domain and a specified domain; and

an update means for updating a parameter of the generation means based on the difference.

### Supplementary note 2

The domain extension learning device according to supplementary note 1, wherein

the generation means generates the pseudo medical examination data from random noise,

the prediction means predicts a domain from the pseudo medical examination data, and outputs a prediction label and a prediction value, and

the calculation means acquires a specified label and a specified value as the specified domain, and calculates a difference between the prediction label and the specified label and a difference between the prediction value and the specified value.

### Supplementary note 3

The domain extension learning device according to supplementary note 2, wherein

the prediction means includes a recognition means and a regression means,

the recognition means predicts a category variable from the pseudo medical examination data and outputs the prediction label, and

the regression means predicts a continuous variable from the pseudo medical examination data and outputs the prediction value.

### Supplementary note 4

The domain extension learning device according to supplementary note 1, wherein

the generation means generates the pseudo medical examination data based on actual medical examination data and a domain conversion label,

the prediction means outputs prediction domain information from the pseudo medical examination data, and

the calculation means acquires specified domain information that is information regarding a known domain as the specified domain, and calculates a difference between the prediction domain information and the specified domain information.

### Supplementary note 5

The domain extension learning device according to supplementary note 4, wherein

the domain conversion label represents a difference between a domain of target pseudo medical examination data that is a conversion destination and a domain of the actual medical examination data that is a conversion source, and includes an arbitrary number of category variables that are conversion destinations and a difference of at least one continuous variable, and

the domain of the target pseudo medical examination data that is a conversion destination is a known domain.

### Supplementary note 6

The domain extension learning device according to supplementary notes 3 or 5, wherein

the category variable includes at least one of race, gender, and disease, and

the continuous variable includes at least one of age, BMI, and a blood pressure value.

### Supplementary note 7

The domain extension learning device according to supplementary note 5, wherein

the specified domain information is a representative feature amount of a domain of a conversion destination, and

the prediction means extracts a feature amount from the pseudo medical examination data, and outputs the extracted feature amount as the prediction domain information.

### Supplementary note 8

The domain extension learning device according to supplementary note 5, wherein

the specified domain information is a label representing a conversion destination domain, and

the prediction means outputs attribution probability values of a plurality of labels from the pseudo medical examination data, and outputs the attribution probability values as the prediction domain information.

### Supplementary note 9

The domain extension learning device according to supplementary note 1, wherein the generation means comprises a deep learning model.

### Supplementary note 10

A domain extension learning method executed by a computer, comprising:

performing generation processing of generating pseudo medical examination data;

performing prediction processing of predicting a domain from the pseudo medical examination data;

performing calculation processing of calculating a difference between the predicted domain and a specified domain; and

updating a parameter of the generation processing based on the difference.

### Supplementary note 11

A program that causes a computer to execute:

performing generation processing of generating pseudo medical examination data;

performing prediction processing of predicting a domain from the pseudo medical examination data;

performing calculation processing of calculating a difference between the predicted domain and a specified domain; and

updating a parameter of the generation processing based on the difference.

While the present disclosure has been particularly shown and described with reference to example embodiments and examples thereof, the present disclosure is not limited to these example embodiments and examples. It will be understood by those of ordinary skill in the art that various changes in form and details may be made therein without departing from the spirit and scope of the present disclosure as defined by the claims.

### DESCRIPTION OF SYMBOLS

10, 20 learning device

101, 201 pseudo data generation unit

102, 202 domain recognition unit

102a recognition unit

102b regression unit

103, 203 difference calculation unit

104, 204 parameter update unit

1. A domain extension learning device comprising:
at least one memory configured to store instructions; and
at least one processor configured to execute the instructions to:
generate pseudo medical examination data using a model targeted for training;
predict a domain from the pseudo medical examination data;
calculate a difference between the predicted domain and a specified domain; and
update a parameter of the model based on the difference.

2. The domain extension learning device according to claim 1, wherein
the model generates the pseudo medical examination data from random noise,
the one or more processors predict a domain from the pseudo medical examination data, and outputs a prediction label and a prediction value, and
the one or more processors acquire a specified label and a specified value as the specified domain, and calculate a difference between the prediction label and the specified label and a difference between the prediction value and the specified value.

3. The domain extension learning device according to claim 2, wherein the one or more processors are configured to
predict a category variable from the pseudo medical examination data and output the prediction label, and
predict a continuous variable from the pseudo medical examination data and output the prediction value.

4. The domain extension learning device according to claim 1, wherein
the model generates the pseudo medical examination data based on actual medical examination data and a domain conversion label,
the one or more processors output prediction domain information from the pseudo medical examination data, and
the one or more processors acquire specified domain information that is information regarding a known domain as the specified domain, and calculate a difference between the prediction domain information and the specified domain information.

5. The domain extension learning device according to claim 4, wherein
the domain conversion label represents a difference between a domain of target pseudo medical examination data that is a conversion destination and a domain of the actual medical examination data that is a conversion source, and includes an arbitrary number of category variables that are conversion destinations and a difference of at least one continuous variable, and
the domain of the target pseudo medical examination data that is a conversion destination is a known domain.

6. The domain extension learning device according to claim 3, wherein
the category variable includes at least one of race, gender, and disease, and
the continuous variable includes at least one of age, BMI, and a blood pressure value.

7. The domain extension learning device according to claim 5, wherein
the specified domain information is a representative feature amount of a domain of a conversion destination, and
the one or more processors extract a feature amount from the pseudo medical examination data, and outputs the extracted feature amount as the prediction domain information.

8. The domain extension learning device according to claim 5, wherein
the specified domain information is a label representing a conversion destination domain, and
the one or more processors output attribution probability values of a plurality of labels from the pseudo medical examination data, and outputs the attribution probability values as the prediction domain information.

9. A domain extension learning method comprising:
generating pseudo medical examination data using a model targeted for training;
predicting a domain from the pseudo medical examination data;
calculating a difference between the predicted domain and a specified domain; and
updating a parameter of the model based on the difference.

10. A non-transitory computer-readable recording medium recording a program for causing a computer to execute processing comprising:
generating pseudo medical examination data using a model targeted for training;
predicting a domain from the pseudo medical examination data;
calculating a difference between the predicted domain and a specified domain; and
updating a parameter of the model based on the difference.

11. The domain extension learning device according to claim 1, wherein the model comprises a deep learning model.
